(19) United States
(12) Patent Application Publication
(10) Pub. No.: US 2011/0218517 A1
(43) Pub. Date: Sep. 8, 2011

(54) IN VIVO CHEMICAL STABILIZATION OF VULNERABLE PLAQUE

(76) Inventors: Matthew F. Ogle, Fitchburg, WI (US); Eric J. Dippel, Bettendorf, IA (US)

(21) Appl. No.: 12/900,906

(22) Filed: Oct. 8, 2010

Related U.S. Application Data
(60) Provisional application No. 61/250,040, filed on Oct. 9, 2009.

(51) Int. Cl. A61M 31/00; A61M 25/10; A61K 38/18; A61P 9/00

(52) U.S. Cl. 604/508; 604/103.01; 514/8.1

(57) ABSTRACT

Procedures for the stabilization of vulnerable plaque can comprise identification of the vulnerable plaque and the delivery of a stabilization composition to the location of the vulnerable plaque. For example, the stabilization composition can be delivered to an isolated portion of the vessel with the vulnerable plaque and/or alternatively or additionally using a profusion balloon that delivers the stabilization composition at the vessel wall near the vulnerable plaque. Suitable stabilization compositions can comprise a collagen crosslinking agent, an elastin stabilization agent, VEGF or a combination thereof. Vascular endothelial growth factor (VEGF) can be used to encourage endothelialization of the vessel wall near the vulnerable plaque.

### CROSS REFERENCE TO RELATED APPLICATIONS

This application claims priority to U.S. provisional patent application Ser. No. 61/250,040 filed on Oct. 9, 2009 to Ogle et al., entitled “In Vivo Chemical Stabilization of Vulnerable Plaque,” incorporated herein by reference.

### FIELD OF THE INVENTION

The inventions, in general, are related to compositions for the stabilization of vulnerable plaque in blood vessels. The inventions are further related to methods for the stabilization of vulnerable plaque and to devices used for the delivery of stabilization compositions and the like to the location of the vulnerable plaque.

### BACKGROUND

Vulnerable plaque generally refers to an unstable structure comprising in part a collection of white blood cells, lipids and cholesterol along the walls of an artery. The body's normal reaction to injury involves inducing swelling or inflammation at the affected area. However, that inflammation also may occur within the walls of the arteries. This inflammation can lead to the formation of “soft” or vulnerable plaque, which includes not only the debris that clogs an artery, but also various cell types whose function is to assist with the clotting of the blood. Due the instability of the plaque, vulnerable plaque is associated with sudden adverse events, such as heart attacks or strokes.

### SUMMARY OF THE INVENTION

In a first aspect, the invention pertains to a method for the treatment of vulnerable plaque comprising delivering a first stabilization composition to stabilize vulnerable plaque within an isolated section of a blood vessel at a location including identified vulnerable plaque. The composition can comprise an elastin stabilization agent, a collagen stabilization agent, VEGF, or a combination thereof.

In another aspect, the invention pertains to a composition comprising a biocompatible carrier, a collagen crosslinking agent and a VEGF growth factor, wherein composition has a concentration of the collagen crosslinking agent at least about 0.5 w/v %. A method for the stabilization of vulnerable plaque can comprise the delivery of the composition to a location in a vessel with vulnerable plaque.

In a further aspect, the invention pertains to a medical device for delivery of a composition at a location in a blood vessel in which the device comprises: a shaft comprising a balloon lumen, a permeable balloon supported by the shaft with an interior in fluid communication with the balloon lumen, and a reservoir holding a stabilization agent near the distal end of the shaft and being configured for controlled delivery of the stabilization agent to the balloon through the balloon lumen, wherein the stabilization agent comprises an elastin stabilization agent, a collagen stabilization agent, VEGF, or a combination thereof. A method of stabilization of vulnerable plaque can comprise the use of this permeable balloon device for the delivery of a stabilization agent to a location with vulnerable plaque.

In an additional aspect, the invention pertains to a medical device comprising a vascular isolation apparatus and a reservoir of VEGF. The vascular isolation device can comprise a sealing element operably connected with a catheter having a lumen with fluid passage between the reservoir and a fluid delivery port. In general, the sealing member can be configured for isolating a portion of a blood vessel when the catheter is delivered in a less invasive endovascular procedure, and wherein the fluid delivery port is positioned for delivery of fluid to the isolated portion of the blood vessel.

### BRIEF DESCRIPTION OF THE DRAWINGS

FIG. 1 is a schematic sectional view of a portion of a artery wall with a vulnerable plaque.

FIG. 2 is a side perspective view of a device for isolating a portion of a blood vessel in which some portions of the device are transparent to provide for visualization of some hidden elements.

FIG. 3 is a schematic fragmentary side view of a profusion balloon catheter for delivery of a stabilization composition.

FIG. 4 is a fragmentary schematic view of a delivery device being positioned within a vessel near a location of vulnerable plaque.

FIG. 5 is a fragmentary side view of a delivery device with a sealing element positioned at a location of vulnerable plaque.

FIG. 6A is a fragmentary side view of the delivery device of FIG. 5 in an expanded configuration.

FIG. 6B is a section view of a fluid exchange element of the delivery device of FIG. 6A taken at line B-B of FIG. 6A.

FIG. 7 is a fragmentary side view of a profusion balloon expanded at the position of vulnerable plaque within a vessel.

### DETAILED DESCRIPTION OF PREFERRED EMBODIMENTS

The approaches described herein are directed to stabilizing the vulnerable plaque structure and/or to encourage remodeling of the vessel wall to form a healthier and more stable vessel. In particular, the plaque can be treated to reduce the likelihood of subsequent rupture. If the plaque has ruptured, stabilization approaches can be used that also tend to down regulate the inflammatory response. Suitable devices have been developed for the localized delivery of desired composition within an isolated portion of a vessel such that the composition is maintained within the selected portion of the vessel for a selected period of time. In particular, stabilization agents can be used within localized segments of the blood vessel to stabilize fibrous structural proteins, such as elastin and or collagen, generally through the formation of chemical crosslinks. Suitable protein crosslinking agents include, for example, phenolic compounds, such as PGG. Some phenolic compounds are particularly effective at crosslinking elastin. The elastin stabilizing agents can also be effective at down regulating the inflammatory response. Similarly, collagen crosslink agents can be delivered to stabilize the vulnerable plaque, which have significant amounts of collagen. Furthermore, chemical agents can be applied that encourage the formation of healthier vessel tissue that are less sensitive to processes resulting in plaque formation as well as destabilization of the plaque. For example, vascular endothelial growth factors can be delivered to the vulnerable plaque to encourage the proliferation of endothelial cells that are found in healthy blood vessel linings.

The devices, compositions and associated procedures described herein are directed to decreasing risks from vulnerable plaque in patients, generally human patients, although these techniques can also be used for farm animals, pets and other mammals. Generally, individuals can be identified using appropriate predictive and diagnostic techniques to identify the vulnerable plaque, and the approaches herein can then be implemented. The stabilization of vulnerable plaques can be combined with mechanical techniques to open restricted vessels and/or pharmaceutical treatments of the vessel within localized segments of the vessel.

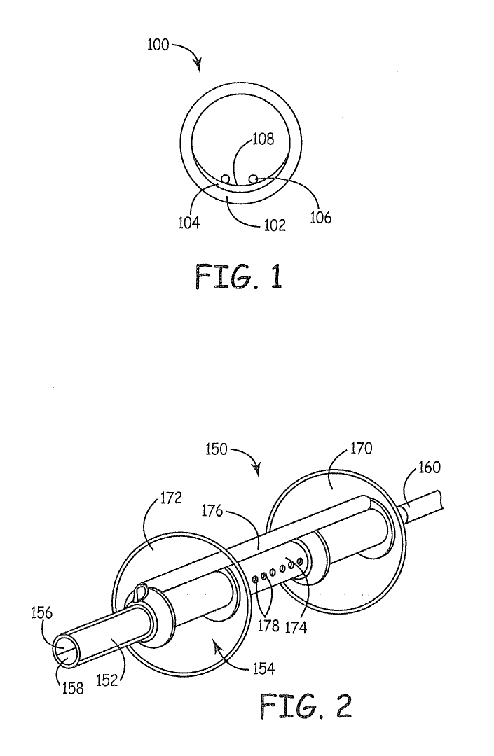

It is believed that vulnerable plaque is formed in an inflammatory process. In this process, fat droplets are absorbed by the artery, which may cause the release of proteins called cytokines that can lead to inflammatory response. The cytokines induce attraction of immune cells, in particular monocytes, to squeeze into the artery walls. In the artery walls, the monocytes can mature into macrophages, which can absorb the fat droplets. The fat filled cells form a plaque with a thin fibrous coating. When the inflammation is combined with other stresses, such as high blood pressure, the thin fibrous covering over the plaque can crack and bleed, which can result in the spilling of the contents of the vulnerable plaque into the blood stream. The cytokines on the artery walls can result in the capture of blood cells, such as platelets. Platelets are attracted to the site of injury. When the cells clump together, the clump can form a clot large enough to block the artery. Rupture of vulnerable plaques is a significant cause of acute coronary syndrome and myocardial infarctions. A site 100 in a vessel with vulnerable plaque is shown schematically in FIG. 1. Vessel 102 has vulnerable, i.e., soft, plaque 104 along the wall of the vessel. Monocytes 106 are shown at the vulnerable plaque 104. Thin layer 108 covers the surface of the soft, vulnerable plaque 104.

Identification of vulnerable plaques can be an important step toward the development of treatment modalities to stabilize the plaques. Several diagnostic methods are currently available to facilitate detection of vulnerable plaques. Angiography involving visualization of the vessel wall, although with limited discrimination of the plaque. Intravascular ultrasound is an imaging technique that can provide a virtual histology of the vessel tissue for the identification and characterization of vulnerable plaque. Thermography can use temperature imaging to assess the character of plaque. Also, some non-invasive imaging techniques are being explored for ability to identify vulnerable plaque, as discussed further below.

Existing clinical approaches for the treatment of vulnerable plaque have generally related to systemic treatment that promote general arterial health and for general reduction of inflammation. Suitable drugs include, for example, thrombolytic agents, ACE inhibitors, calcium agonists, lipid modifying agents and the like. Localized delivery of drugs using needles, gel paving, porous balloons or drug eluting stents is described briefly in very general terms by Keriakes in an article entitled “In search of vulnerable plaque,” Circulation (2003) 107: 2067-2077, incorporated herein by reference. As described below, the specific stabilization approaches described herein for vulnerable plaque can be combined with drug treatments.

As described herein, desired stabilization compositions can be locally delivered through localized placement of a stabilization composition, which may or may not involve isolation of a portion of a blood vessel. Furthermore, the area of local delivery can be exposed to the stabilization compositions for a desired period of time. For example, the stabilization composition can be delivered through a leaky balloon, an eluting stent or gel paving. These approaches do not isolate a portion of the blood vessel, so degree of release of the stabilization composition should be tolerated by the patient.

In some embodiments, the stabilization compositions can be delivered with devices that isolate a portion of the vessel. After the passage of the selected time period, the remaining composition within the isolated portion of the vessel can be removed and optionally replaced with another stabilization composition, a therapeutic agent, a rinsing composition, the patient's blood, combinations thereof, or the like. The process can be repeated as desired with additional replacement liquids. In particular, protein crosslinking agents can be used to stabilize the plaque. Suitable crosslinking agents generally have multiple functional groups that can bond with protein functional groups. The plaque significantly comprises collagen, and protein crosslinking agents can stabilize the collagen and thereby the plaque with respect to fracture, although crosslinking agents that are particularly effective for crosslinking elastin can also be effective with respect to mechanical stabilization and down regulating an inflammatory response.

Also, it can be desirable to encourage the return of a normal vascular tissue structure along the vessel wall. In healthy vascular tissue, a thin layer of endothelial cells line the vessel over a fibrous extracellular matrix with viable cells, e.g., fibroblast cells. Therefore, the encouragement of endothelial cells can promote a healthy vessel lining. Vascular endothelial growth factors (VEGF) have been identified as relatively specific growth factors for the proliferation of endothelial cells. As used herein, VEGF refers broadly to VEGF proteins, active fragments thereof, active modified versions thereof, purified natural VEGF proteins, recombinant VEGF and the like. VEGF for stimulating endothelial cell colonization has been described further in U.S. Pat. No. 7,722,671 to Carlyle et al., entitled “Medical Devices With Associated Growth Factors,” incorporated herein by reference.

In some embodiments, the stabilization compositions can comprise a protein crosslinking agent, a VEGF growth factor, or combinations thereof as well as a biocompatible carrier, such as sterile buffered saline. The stabilization composition can optionally further comprise a pharmaceutical composition, although a pharmaceutical composition can be incorporated into a distinct therapeutic composition for separate delivery to vulnerable plaque. In general, a protein crosslinking agent bonds with protein functional groups. While this bonding may have a significant non-specific component, a protein crosslinking agent may be understood to be particularly effective with respect to certain protein while providing reasonable crosslinking or other proteins, such as other structural protein, although the crosslinking agent may be identified by its particularly effective target. Suitable general protein crosslinking agents for general crosslinking of structural protein of the extracellular matrix include, for example, elastin crosslinking agents, such as PGG, collagen crosslinking agents, such as glutaraldehyde or a combination thereof. In some embodiments, a photoactivated crosslinking agent can be used. Detailed discussion of formulations and compositions for connective tissue stabilization through the use of crosslinking agents are disclosed in U.S. Pat. No. 7,713,543 (the '543 Patent) to Vyavahare et al., entitled “Elastin Stabilization of Connective Tissue”, published U.S. patent application 2010/0119605 (the '605 Application) to Isenburg et al., entitled “Compositions for Tissue Stabilization”, and published U.S. patent application 2009/0214654 to Isenburg et al., entitled “Treatment of Aneurysm with Application of Connective Tissue Stabilization Agent in Combination with a Delivery Vehicle,” all three of which are incorporated herein by reference.

As described in the '543 patent cited above, tissue with an elastin component can be strengthened with a phenolic compound, although these compounds can also be used to crosslink tissue that is more predominantly collagen, such as scar tissue of a vulnerable plaque. In particular, it is believed that any of a number of natural and synthetic phenolic compounds can bind structural protein and thereby strengthen the corresponding tissue, such as connective tissue and/or scar tissue, with particularly good bonding to elastin present in connective tissue. In some embodiments, protein crosslinking phenolic compounds include, for example, any compound that comprises at least one phenolic group bound to a hydrophobic core. While not wishing to be bound by theory, it is believed that interaction between the phenolic compound and elastin proteins have aspects involving both the hydroxyl group as well as the hydrophobic core of the molecules. In particular, the large hydrophobic regions of the elastin protein, which are believed to contain sites susceptible to elastase-mediated cleavage, are also believed to contain sites of association between the hydrophobic core of the phenolic compound and the protein. Thus, the association of the hydrophobic core of the phenolic stabilization compound with the hydrophobic region of the elastin may contribute to inhibition of elastin cleavage by elastase. In certain embodiments, the phenolic compounds can comprise one or more double bonds, with which the phenolic compounds can covalently bind to the structural protein, forming an even stronger protective association, between the phenolic compound and the extracellular matrix of the tissue. Phenols with double bonds can be particularly desirable for crosslinking collagen.

Additionally, collagen crosslinking/stabilization compositions have been found to provide a high degree of stabilization of connective tissues, as described in the '605 application cited above. Multi-functional reagents, such as glutaraldehyde, diamines, genipin, acyl azide, and epoxyamines, are known to cross-link functional groups in collagen thereby tissue having a collagen component. In some embodiments, a collagen crosslinking/stabilization agent can be effectively combined with an elastin stabilizing agent. The combination of treatment agents can be contacted with the tissue simultaneously or sequentially.

If VEGF is delivered with a protein crosslinking agent, in some embodiments, the two compositions can be mixed a short time before their use, and/or in further embodiments the two compositions can be mixed during their delivery into the vessel. Mixing of VEGF with a protein crosslinking agent at or near the time of use can reduce the formation of agglomerates resulting from crosslinking of the VEGF with the protein crosslinking agent. In some embodiments, VEGF and a protein crosslinking agent can be sequentially delivered to a portion of the vessel to assist with the stabilization of vulnerable plaque.

Also, the stabilization compositions can comprise pharmaceutical compositions that can be locally administered possibly at higher concentrations than would be delivered systemically. In particular, suitable pharmaceutical compositions include, for example, drugs that are generally given orally, intravenously, intramuscular injection or the like for the treatment of vulnerable plaque. For example, suitable pharmaceutical agents can include, for example, thrombolytic (antithrombic) agents, ACE inhibitors, calcium antagonists, such as amlodipine, lipid modifying agents and the like.

Intravascular devices have been developed that provide for the localized delivery within a blood vessel. A first class of device is designed to seal two cross sections of a blood vessel while providing fluid exchange with an isolated volume thus formed including the walls of the blood vessel between the sealed cross sections. In some embodiments, the devices can also provide for shunted blood flow past the sealed volume. Since blood flow is not occluded downstream from the treatment region, the time constraints on the procedure are not necessarily severe. Several different designs have been developed for this type of device that isolated a portion of a blood vessel while allowing for fluid exchange. A single sealing element or a plurality of sealing elements can be used to provide the seals between the two cross sections of the selected blood vessel. These devices are described further in copending U.S. patent application publication number 2010/0016833 (the '833 application) to Ogle et al., entitled “Devices for the Treatment of Vascular Aneurysm,” incorporated herein by reference.

Another type of device has been developed that provides for the selection of adjustable volumes for the isolated region of the blood vessel. The device comprises two sealing elements supported on different structures. Specifically, a proximal sealing element can be supported on an introducer, and a distal sealing element can be supported on a catheter in which the introducer and the catheter can be moved to adjust their relative longitudinal positions. The devices of this type are particularly suitable for use in arteries below the renal arteries in which the blood flow can be occluded for a significant period of time, generally roughly on the order of several hours, without significant detrimental effects to the patient. The devices can incorporate balloon designs for the sealing elements that provide a strong force, but provide less pressure to reduce the risk of damaging the vessel wall or plaque located at or near the sealing elements. These devices are described further in copending U.S. patent application Ser. No. 12/581,311 (the '311 application) to Ogle et al., entitled “Vascular Medical Devices with Sealing Elements and Procedures for the Treatment of Isolated Vessel Sections,” incorporated herein by reference.

Alternatively or additionally, a profusion balloon can be used for localized delivery of a stabilization composition without isolating a portion of the vessel. In principle, a profusion catheter can be used to deliver a composition in the vicinity of identified vulnerable plaque. However, a profusion balloon has an advantage over a catheter of delivering the composition at the wall of the blood vessel so that the localized concentration at the vessel wall can be correspondingly greater than in the concentration in the downstream vessel. Also, the balloon can also provide some mechanical opening of the vessel lumen if the vulnerable plaque is initially constricting the vessel to some degree.

The in vivo delivery of stabilization compositions to vulnerable plaque provides a direct approach to the stabilization of vulnerable plaque. The stabilization process can comprise mechanical stabilization, down regulation of the inflammatory response and/or promotion of healthier vessel structure. Thus, the in vivo treatment processes described herein can provide significant alternatives to systematic treatment and/or additional tools to be combined with systematic treatments. A stabilization composition for the stabilization of vulnerable plaque can comprise an elastin stabilization composition, a collagen stabilizing agent, VEGF, and/or a pharmaceutical adjunctive. The stabilization compositions can be formulated with desirable delivery vehicles that can be formulated in effective concentrations for in vivo direct delivery to the vulnerable plaque. If multiple agents are used with a patient, the agents may be combined within a single formulation and/or distinct formulations can be used for simultaneous and/or sequential delivery.

Thus, the techniques described herein can be effective to provide mechanical stabilization of vulnerable plaque along with biological manipulation of the relevant portions of the tissue. The mechanical stabilization can reduce incidences of near term rupture of the vulnerable plaque, release of thrombus from the vulnerable plaque and/or other adverse effects emanating from the vulnerable plaque. Along with the stabilization, additional treatments can encourage the development of more normal vessel physiology in the longer term.

### Compositions

As noted above, the stabilization of vulnerable plaque can involve the use of one or more stabilization compositions. A stabilization agent generally is within a suitable biocompatible carrier which is suitable for the particular delivery approach. Suitable stabilization agents can include, for example, collagen stabilization agents, elastin stabilization agents, VEGF or combinations thereof. Suitable therapeutic agents can be delivered simultaneously or sequentially with respect to a stabilization agent.

Suitable therapeutic agents or drugs include, for example, thrombolytic agents, ACE inhibitors, calcium agonists, lipid modifying agents, combinations thereof or the like. Lipid modifying agents can include, for example, statins such as atorvastatin, fluvastatin, lovastatin, pitavastatin, pravastatin, rosuvastatin, and simvastatin; niacin; fibrates such as bezafibrate, ciprofibrate, clofibrate, gemfibrozil, and fenofibrate; or combinations thereof. Thrombolytic agents can include, for example, heparin, aspirin, adenosine diphosphate receptor antagonists, Gp IIb/IIIa inhibitors or combinations thereof. Angiotensin converting enzyme (ACE) inhibitors may inhibit oxygen-free radical production and promote normal endothelial function. ACE inhibitors include, for example, captopril, zofenopril, enalapril, ramipril, quinipril, fosinopril and combinations thereof. Systemic and local delivery through a catheter or as a stent coating for pharmaceutical agents is described further in Spratt et al., Heart volume 90 (12), December 2004 (1392-1394), incorporated herein by reference.

A therapeutic composition or stabilization composition can comprise one or more therapeutic agents in some embodiments in a concentration from about 0.0001% to about 10% by weight. For example, a particular selected concentration of an additional agent may be influenced by the delivery method and incubation time. In some embodiments, the compositions can comprise concentrations of the therapeutic agent ranging from about 0.001% to about 7.5% by weight and in additional embodiments from about 0.01% to about 5% by weight. A person of ordinary skill in the art will recognize that additional ranges within the explicit ranges above are contemplated and are within the present disclosure.

Stabilization compositions and/or therapeutic compositions can comprise pharmaceutically acceptable sterile aqueous or nonaqueous solutions, dispersions, suspensions, emulsions or the like. Examples of suitable aqueous and nonaqueous carriers, diluents, solvents or vehicles include water, ethanol, polyols (e.g., glycerol, propylene glycol, polyethylene glycol and the like), vegetable oils (e.g., olive oil), injectable organic esters such as ethyl oleate, or suitable mixtures thereof. In addition, the composition can contain minor amounts of auxiliary substances, such as preservatives, wetting or emulsifying agents, dispersing agents, pH buffering agents, combinations thereof; or the like that can enhance the effectiveness and/or delivery of the stabilizing composition and/or therapeutic composition. The therapeutic compositions should be appropriately sterile at the time of use. Prevention of the action of microorganisms may be enhanced by the inclusion of various antibacterial and antifungal agents such as paraben, chlorobutanol, phenol, sorbic acid and the like. It may also be desirable to include isotonic agents such as sugars, sodium chloride and the like.

A stabilization composition or therapeutic composition can comprise one or more buffers. For example, a composition having a pH from about 4.0 to about 9.0 may be formulated with inclusion of purified water, saline and a biocompatible buffer, such as phosphate buffers, borate buffers, HEPES, PIPES, MOPSO or combinations thereof. In further embodiments, a composition of the invention may be formulated to have a pH of between about 5.5 and about 7.4. A person of ordinary skill in the art will recognize that additional ranges of pH within the explicit ranges above are contemplated and are within the present disclosure.

In some embodiments, the compositions can comprise pharmaceutically acceptable salts of the components therein, e.g., those that may be derived from inorganic or organic acids. Pharmaceutically acceptable salts are well known in the art. For example, S. M. Berge, et al. describes pharmaceutically acceptable salts in detail in J. Pharmaceutical Sciences (1977) 66:1 et seq., which is incorporated herein by reference. As noted above, a suitable rinsing composition to facilitate removal of therapeutic compositions can comprise buffered saline, such as buffered sodium chloride.

### VEGF—Vascular Endothelial Growth Factors

VEGF refers to a family of polypeptides that have been found to preferentially stimulate proliferation of vascular endothelial cells, such as smooth muscle cells. VEGF was originally referred to as vascular permeability factor. Six members of the VEFG family have been identified: VEGF (VEGF-A), placenta growth factor (PIGF), VEGF-B, VEGF-C, VEGF-D and VEGF-E. VEGF-A can take several forms of peptide homodimers or heterodymers, and subunits have been identified with molecular weights of 121, 165, 189 and 206 amino acids each. The different subunits have been identified as originating from different splicing with respect to exons in the VEGF gene. The various VEGF proteins have been identified as promoting angiogenesis and lymph-angiogenesis. The genes encoding the various VEGF proteins have been identified.

As used herein unless otherwise specified, VEGF refers to currently identified and future identified VEGF proteins that selectively promote the chemotaxis and proliferation of endothelial cells, peptide fragments thereof, and functionally active chemically modified versions thereof. Activity of chemically modified versions can be examined in cell culture. VEGF proteins and genes encoding VEGF proteins are described further in U.S. Pat. No. 7,722,671 to Carlyle et al, entitled “Medical Devices With Associated growth Factors,” published U.S. patent application 2010/0041605 to Alitalo et al., entitled “Use of VEGF-C or VEGF-D in Reconstructive Surgery,” both of which are incorporated herein by references, and referenced cited therein.

In some embodiments, a stabilization composition can comprise VEGF in a concentration from about 1 ng/ml to about 10 μg/ml, in further embodiments from about 10 ng/ml to about 5 μg/ml and in other embodiments from about 25 ng/ml to about 1 μg/ml. If the stabilization composition comprises VEGF, the stabilization composition can further comprise a collagen crosslinking agent, such as glutaraldehyde, for example, in a low concentration to reduce crosslinking prior to contact with the tissue. However, the VEGF and collagen crosslinking agent can be combined a relatively short time prior to delivery to the patient so that a higher concentration of crosslinking agent can be used to greater efficacy in an appropriate amount of time within the patient. Thus, in some embodiments, the stabilization composition comprising VEGF can comprise a collagen crosslinking agent in a concentration of at least about 0.5 w/v %, in further embodiments at least about 0.75 w/v % and in additional embodiments from about 1 w/v % to about 10 w/v % collagen crosslinker. A person of ordinary skill in the art will recognize tat additional ranges of concentration within the explicit ranges above are contemplated and are within the present disclosure. Mass to volume percentage is the relationship of a solute to a solvent expressed as grams of solute per milliliter of the total solution, as used herein and as standard in the particular art.

### Collagen Crosslinking

With respect to providing mechanical stabilization of vulnerable plaque, collagen crosslinking/stabilization compositions have been found to provide a high degree of stabilization of connective tissues, as described in the '605 application cited above. In some embodiments, a collagen crosslinking/stabilization agent can be used as a single stabilization agent. In additional or alternative embodiments, a collagen crosslinking agent can be effectively combined with an additional stabilization agent, such as VEGF, an elastin stabilizing agent or a combination thereof. The combination of treatment agents can be contacted with the tissue simultaneously or sequentially.

Multi-functional reagents, such as glutaraldehyde, diamines, genipin, acyl azide, and epoxyamines, are known to cross-link functional groups in collagen thereby tissue having a collagen component. Some known functional groups for collagen cross-linking are amino, thiol, hydroxyl, and carbonyl in collagen and/or other proteins, such as other structural proteins. By binding to and crosslinking collagen and/or other proteins, the multi-functional agents can increase the mechanical strength of the tissue to reduce the risk of rupture of vulnerable plaque. Collagen containing tissue treated with collagen crosslinking/stabilization agent with or without combination with elastin stabilization agent may exhibit enhanced rupture resistance, resistance to enzymatic degradation such as elastase and collagenase, and a higher thermal denaturation temperature. In the case of vulnerable plaque, the increased mechanical strength of the tissue can correspondingly reduce the risk of rupture of the plaque, which can result in further scarring and/or the release of emboli.

Glutaraldehyde and other multi-functional aldehyde compounds are known to bind to and stabilize collagen in a collagen containing tissue. Glutaraldehyde in particular self-polymerizes to form polymer chains that are believed to be effective at crosslinking between collagen fibers. Glutaraldehyde polymerizes with itself and/or with nearby active groups from collagen and/or other proteins creating crosslinks in the treated tissue. However, residual unreacted free aldehyde groups from glutaraldehyde can contribute with regards to toxicity and calcification. Treatment of bioprosthetic tissue to reduce toxicity is described in U.S. Pat. No. 6,471,723 to Ashworth et al., entitled “Biocompatible Prosthetic Tissue,” incorporated herein by reference.

Difunctional aldehydes, e.g., glutaraldehyde, have been used for some time to fix colleganous tissue, such as bovine pericardium, for the preparation of bioprosthetic materials, but alternative collagen crosslinking agents have been proposed. One of the alternative collagen stabilizing agents comprises diamines, generally with at least two free primary amine groups, such as 1,6-hexanediamine and 1,7-heptanediamine. The diamines bond to carboxyl groups in proteins to form a crosslinked structure. It has been found that coupling agents and coupling enhancers facilitate this crosslinking/stabilization process with diamines. For example, suitable coupling agents include carbodiimides, such as 1-ethyl-3-(3-dimethyl aminopropyl)-carbodiimide (EDC) and/or N-hydroxysuccinimide (NHS). The carbodiimides function as a coupling agent in the crosslinking/stabilization reaction, and are generally used along with a coupling enhancer. For example, EDC can be used in conjunction with N-hydroxysulfosuccinimide (Sulfo-NHS), which acts as an enhancer to the reaction. Other suitable coupling enhancers include, for example, N-hydroxybenzotriazole (HOBt), N,N-dimethyl-4-aminopyridine (DMAP) and N-hydroxysuccinimide. By coupling the amine and carboxyl groups within the tissue, this treatment creates amide bonds or bridges between and/or inside proteins, thus crosslinking the tissue. In vitro crosslinking of bioprosthetic tissue with diamines along with coupling agents and/or coupling enhancers is described further in published U.S. patent application 2006/0159641A to Girardot et al., entitled “Variably Crosslinked Tissue,” incorporated herein by reference. Collagen stabilization can be achieved using other active agent or alternative methods.

For example, genipin is a naturally occurring plant compound capable of crosslinking collagen, and epoxy compounds have reactive functional groups that are reactive with several functional groups found in proteins, such epoxies can be used to crosslink proteins, especially collagen, within tissue. Additionally, epoxy amine polymer compounds are also suitable collagen crosslinking agents that are described further in U.S. Pat. No. 6,391,538 to Vyavahare et al., entitled “Stabilization of Implantable Bioprosthetic Tissue,” incorporated herein by reference. An example of a poly-epoxyamine compound suitable as a collagen crosslinking agent is triglycidylamine, a triepoxy amine. The use of triglycidyleamine in stabilization of bovine pericardium tissue described in Connolly et al. Am J. Pathol. 2005, 166(1): 1-13 entitled “Triglycidylamine crosslinking of porcine aortic valve cusps or bovine pericardium results in improved biocompatibility, biomechanics, and calcification resistance,” incorporated herein by reference, can similarly be adapted for the use in heart tissue. Moreover, free carboxyl groups on collagen can be converted into acyl azide groups, which react with free amino groups on adjacent side chains to crosslink the collagen tissue. This crosslinking approach is described in Petite et al. Biomaterials 1995; 16(13): 1003-1008, incorporated herein by reference.

Photoactivated crosslinking agents may be desirable for in vivo delivery of collagen stabilization agents for certain applications, such as the stabilization of vulnerable plaque, since greater control can be applied to the crosslinking process. Photo-oxidation fixation can be based on the use of a photoactive dye as a catalyst. Suitable dyes include, for example, PhotoFix™ used by Carbomedics for bioprosthetic heart valves, methylene blue, methylene green, rose bengal, riboflavin, proflavin, fluorescein, eosin, pyridoxal-5-phosphate, or combinations thereof. The catalyst aids in the conversion of amino acids within the tissue, subsequently allowing for crosslink formation between the converted amino acid and nearby amino acids, thus stabilize the tissue. See, for example, the following articles: Adams A K, Talman E A, Campbell L, et al. J Biomed Mater Res 2001; 57(4): 582-587 and Meuris B, Phillips R, Moore M A, et al. Artif Organs 2003; 27(6): 537-543), both of which are incorporated by reference.

Methods for ex vivo crosslinking of collagen with a photocatalyst are described further in U.S. Pat. No. 5,147,514 to Mechanic et al, entitled “Process for cross-linking collagenous material and resulting product,” incorporated herein by reference can similarly be adapted for the stabilization of vulnerable plaque. Photo-oxidation requires exposure of the dye treated tissue to a light source. In the case of vulnerable plaque stabilization, such light source can be supplied by for example, optical fiber. The use of a photoactivated process provides for greater control of the crosslinking reaction since migration of chemicals from the delivery location does not result in crosslinking unless photoactivated. Additionally, oxygen, which may need to be supplied to the therapeutic composition to facilitate the completion of the photo-oxidative reaction, can be supplied with the therapeutic composition.

In some embodiments, the one or more collagen crosslinking/stabilization agents can be provided as a biocompatible composition. For instance, compositions disclosed herein can comprise one or more collagen crosslinking/stabilization agent in a concentration that can extend over a wide range, with a selected concentration generally depending on the particular application, the delivery site targeted by the one or more collagen crosslinking/stabilization agent and the mode for the delivery process. For example, a stabilization composition can comprise one or more collagen crosslinking/stabilization agents at a concentration from about 0.0001 w/v % to about 10 w/v, in further embodiments from about 0.01 w/v to about 5 w/v %, in other embodiments from about 0.1 to about 2.5 w/v %. A person of ordinary skill in the art will recognize that additional ranges of concentration within the explicit ranges above are contemplated and are within the present disclosure. For example, actual concentrations used may be influenced by the particular procedure, size of the targeted area, desired incubation time, and selected pH, in addition to delivery mode, as mentioned above. A person of ordinary skill in the art will recognize that additional ranges of concentration within the explicit ranges above are contemplated and are within the present disclosure.

### Elastin Crosslinking

As described above in the '543 patent, tissue with an elastin component can be strengthened with a phenolic compound, although these compounds can also be used to crosslink a broader range of tissue based on more general protein binding as well as binding to tissues with lower amounts of elastin. As described herein, the elastin crosslinking compounds are used to stabilize tissue that more predominantly comprises collagen, such as scar tissue of a vulnerable plaque. In particular, it is believed that any of a number of natural and synthetic phenolic compounds can bind structural protein and thereby strengthen the corresponding tissue, such as connective tissue and/or scar tissue. In some embodiments, protein crosslinking phenolic compounds include, for example, any compound that comprises at least one phenolic group bound to a hydrophobic core. While not wishing to be bound by theory, it is believed that interaction between the phenolic compound and elastin proteins have aspects involving both the hydroxyl group as well as the hydrophobic core of the molecules. In particular, the large hydrophobic regions of the elastin protein, which are believed to contain sites susceptible to elastase-mediated cleavage, are also believed to contain sites of association between the hydrophobic core of the phenolic compound and the protein. Thus, the association of the hydrophobic core of the phenolic stabilization compound with the hydrophobic region of the elastin may contribute to inhibition of elastin cleavage by elastase. In certain embodiments, the phenolic compounds can comprise one or more double bonds, with which the phenolic compounds can covalently bind to the structural protein, forming an even stronger protective association between the phenolic compound and the extracellular matrix of the tissue. Phenols with double bonds can be particularly desirable for crosslinking collagen.

Suitable phenolic compounds with one or more phenol groups extending from the hydrophobic core of the molecule can include, but are not limited to, flavonoids and their derivatives (e.g., anthocyanins, quercetin), flavolignans, phenolic rhizomes, flavan-3-ols including (+)-catechin and (−)-epicatechin, other tannins and derivatives thereof (such as tannic acid, pentagalloylglucose, nobotanin, epigallocatechin gallate, and gallotannins), ellagic acid, procyanidins, and the like. Suitable phenolic compounds include synthetic and natural phenolic compounds. For example, natural phenolic compounds can include those found in extracts from natural plant-based sources such as extracts of olive oil (e.g., hydroxytyrosol (3,4-dihydroxyphenylethanol) and oleuropein, extracts of cocoa bean that can contain epicatechin and analogous compounds, extracts of Camellia including C. senensis (green tea) and C. assaimic, extracts of licorice, sea whip, aloe vera, chamomile, and the like.

In some embodiments, the phenolic compounds can be tannins and derivatives thereof. Tannins can be found in many plant species. For example, the tea plant (Camellia sinensis) has a naturally high tannin content. Green tea leaves are a major plant source of tannins, as they not only contain the tannic and gallic acid groups, but also prodelphinidin, a proanthocyanidin. Tannins are also found in wine, particularly red wine as well as in grape skins and seeds. Pomegranates also contain a diverse array of tannins, particularly hydrolysable tannins.

Pentagalloylglucose (PGG) and tannic acid (TA) are members of the tannin family, a group of naturally derived polyphenolic compounds. PGG is a less toxic derivative of tannic acid. PGG is naturally occurring, relatively non-toxic and not expected to exhibit significant side effects. PGG is characterized by a D-glucose molecule esterified at all five hydroxyl moieties by gallic acid (3,4,5-trihydroxybenzoic acid). In general, it is understood that the PGG molecule can have 1-4 galloyl group(s) and the galloyl groups can assume different stereo chemical forms. For example, PGG can be in either alpha or beta forms. The '543 patent reported that periarterial treatment with PGG preserves elastin fiber integrity and hinders aneurysmal dilatation of the abdominal aorta in a clinically relevant model of aortic aneurysms.

In general, the phenolic compounds described herein can be provided in a biocompatible stabilization composition. For instance, compositions disclosed herein can comprise one or more phenolic compounds in a concentration that can vary over a wide range, with a suitable concentration generally depending on the particular application, the delivery site targeted by the phenolic compound and the mode of delivery. For example, in some embodiments, a composition can comprise one or more phenolic compounds at a concentration from about 0.0001 w/v % to about 10 w/v %, in further embodiments from about 0.01 w/v % to about 7.5 w/v % and in additional embodiments from about 0.05 w/v % to about 5 w/v %. A person of ordinary skill in the art will recognize that additional concentration ranges within the explicit ranges herein are contemplated and are within the present disclosure. Some concentrations reported herein are weight/volume percentages. Mass to volume percentage is the relationship of a solute to a solvent expressed as grams of solute per milliliter of the total solution, as used herein and as standard in the particular art. For example, 0.06 g of pentagalloylglucose (PGG) in 100 mL of solution is considered a 0.06% w/v PGG solution.

For stabilization compositions that are expected to impart a fast and strong binding thus stabilization effect, relatively high concentration of elastin stabilization agents maybe used. Toxicity reduction in these relatively high concentration cases can be especially of interest. For example, when tannin family of compounds such as PGG or TA is used, it can be desirable to maintain low level of free or unbound gallic acid in the composition. In some embodiments, the composition can comprise little or no unbound gallic acid. In some embodiments, the composition can comprise no more than about 5 wt % un-bound gallic acid. In further embodiments, the composition can comprise no more than about 2.5 wt % unbound gallic acid and in additional embodiments no more than about 1 wt % unbound gallic acid A person of ordinary skill in the art will recognize that additional ranges of concentration within the explicit ranges above are contemplated and are within the present disclosure.

### Rinsing Compositions

In some embodiments, it is desirable to rinse an isolated portion of a blood vessel following completion of contact with a stabilization composition. Rinsing compositions can comprise a simple rinse that is intended to remove unreacted stabilization compositions through dilution and physical fluid exchange. In some embodiments, a rinse agent can comprise a composition that decreases any residual toxicity associated with the treatment agent. In some procedures, a plurality of rinsing agents can be used in a selected order with respect to each other and with respect to the application of stabilization compositions. The volume of rinsing agent can be selected to achieve the desired rinsing effect.

In some embodiments, a rinsing agent can be sterile saline, e.g. water with sodium chloride, at or near physiological ionic strength, e.g., about 0.9% w/v NaCl. Saline can optionally include a suitable buffer. Physiologically acceptable buffers are known in the art, such as phosphate salts, carbonate salts, TRIS (tris(hydroxymethyl)methylamine), MOPS (3-(N-morpholino)propanesulfonic acid), MES (2-(N-morpholino)ethanesulfonic acid) or tricine (N-tris(hydroxymethyl)methylglycine). Suitable sterile salines are available commercially for intravenous use that can be adapted as rinsing agents.

Following treatment with aldehyde crosslinking agents, the unreacted free aldehyde groups on the treated tissue can be further treated with compounds that are reactive amines such as lysine or glutamine, thus lessening the impact of potential aldehyde cytotoxicity. See, Simionescu A, et. al., R. J Biomed Mater Res 1991; 25(12): 1495-1505 and Jorge-Herrero E, et al. Biomaterials 1996; 17(6): 571-575, both of which are incorporated herein by reference. Other amines that can be used including other amino acids as well as diamines, such as 1,6 hexane diamine or N,N,N′,N′-tetramethyl diamine. Alternatively, aldehyde groups from a collagen crosslinking/stabilization agent can be quenched with organic or inorganic agents disclosed in U.S. Pat. No. 6,471,723 to Ashworth et al., entitled “Biocompatible Prosthetic Tissue,” incorporated herein by reference. Ashworth et al. discovered that improved reduction in cytotoxicity could result from the use of a plurality of toxicity reducing agents.

In addition to amines, suitable toxicity reducing agents can include, for example, inorganic sulfur-oxygen containing anions, organic sulfates, ammonium salts and surfactants. Suitable inorganic sulfur-oxygen anions include, for example, sulfate anions SO₄⁻², thiosulfate anions S₂O₄⁻², and protonated forms thereof, such as bisulfate HSO₄⁻. Suitable organic sulfates include, for example, aliphatic sulfates, such as methyl sulfate CH₃O₄S⁻, dimethyl sulfate (CH₃)₂O₄S and dodecyl sulfate CH₃(CH₂)₁₁O₄S⁻ as well as protonated forms thereof. Ammonium cations can be introduced, for example, as ammonium chloride, ammonium hydroxide or other suitable ammonium salt.

Suitable surfactants include, for example, nonionic surfactants, such as aliphatic fatty acid esters, polypropyleneglycol fatty acid esters, e.g., propyleneglycol monostearate, and glycerol fatty acid esters, e.g., glycerol monostearate. Other suitable nonionic surfactants include, for example, polyalkylene ethers, such as polyethylene lauryl ether, polyoxyethylene oleyl ether, polyoxyethylene cetyl ether, and polyethylene glycol p-isooctyl phenyl ethers, such as Triton™ X-100 and the like. Furthermore, suitable nonionic surfactants include, for example, polyoxyethylene sorbitan esters that include, for example, polyoxyethylene compounds in an ether linkage with sorbital and an ester linkage with a fatty acid, such as Polysorbate 80 or polyethylene (2) sorbitan monooleate, which is sold under the trade name Tweene 80. Other nonionic surfactants are used for medical applications, which are suitable for rising agents.

Toxicity reducing agents are generally dissolved into an aqueous solution for use. The solution can be buffered, for example, using a physiological buffer described above. In some embodiments, the toxicity reducing agents can be included in a concentration from about 0.005 molar (M) to about 3 M, and in further embodiments from about 0.01M to about 1M. A person of ordinary skill in the art will recognize that additional ranges of concentrations within the explicit ranges above are contemplated and are within the present disclosure. As noted above, it can be desirable to include two different classes of toxicity reducing agents within the classes of agents described above. In particular, Ashworth et al., above, suggest the use of a combination of an amine, a sulfate, ammonium ions and a surfactant.

### Delivery Vehicle

As described herein, stabilization compositions can be delivered in various formats. In some formats, the stabilization agents are delivered in a format for rapid efficacy since the treatment time is limited. In other embodiments, the stabilization composition can be provided over more extended periods of time. The delivery of stabilization agents, e.g., protein crosslinking agents, using a delivery vehicle, such as for delayed release, is described further in published U.S. patent application 2009/0214654 to Isenberg et al., entitled “Treatment of Aneurysm With Application of Connective Tissue Stabilization Agent in Combination With a Delivery Vehicle,” incorporated herein by reference. In some embodiments, the method can comprise use of timed release or sustained release delivery vehicles. Such systems can be desirable, for instance, in situations where long term delivery of the agents is appropriate and desired.

According to one embodiment, a sustained-release matrix can comprise materials, usually polymers, which are degradable by enzymatic or acid/base hydrolysis or by dissolution. Once located at or near the target tissue, a matrix can be acted upon by enzymes and body fluids. The sustained-release matrix can be chosen from biocompatible materials such as liposomes, polylactides (polylactic acid), polyglycolide (polymer of glycolic acid), polylactide co-glycolide (co-polymers of lactic acid and glycolic acid) polyanhydrides, poly(ortho)esters, polyproteins, hyaluronic acid, collagen, chondroitin sulfate, carboxylic acids, fatty acids, phospholipids, polysaccharides, nucleic acids, polyamino acids, amino acids such as phenylalanine, tyrosine, isoleucine, polynucleotides, polyvinyl propylene, polyvinylpyrrolidone and silicone. Possible biodegradable polymers and their use are described, for example, in detail in Brem et al. (1991, J. Neurosurg. 74:441-6), which is hereby incorporated by reference in its entirety. In some embodiments, the stabilization composition and/or therapeutic composition can be loaded in a drug delivery vehicle via encapsulation, coating, infusion, or any other loading mechanism, such as those known in the art.

In one embodiment, the disclosed agents can be targeted to connective tissue by use of a hydrogel delivery vehicle. Hydrogels herein refer to polymeric matrices that can be highly hydrated while maintaining structural stability. Hydrogels are hydrophilic polymers that do not dissolve in aqueous solution generally as a result of crosslinking. Hydrogel delivery vehicles can include, for example, natural polymers such as glycosaminoglycans, polysaccharides, proteins, and the like, as well as synthetic polymers, as are generally known in the art. Pluronic™ polymers generally comprise polyoxy-propylene/polyoxyethylene block copolymers. Thus, hydrogels from the crosslinking of these block copolymers and similar compositions can be referred to as Pluronic™ hydrogels.

Polymeric particles for drug delivery generally include, for example, biocompatible polymers and may or may not be spherical. The polymeric particles generally have an average particle diameter of no more than about 5 microns, in further embodiments no more than a micron and in additional embodiments no more than about 250 nanometers, where the diameter is an average dimension through the particle center for non-spherical particles. The delivery of drugs using nanoparticles and microparticles is described further in published U.S. Patent application 2006/0034925 to Au et al, entitled “Tumor Targeting Drug-Loaded Particles,” incorporated herein by reference.

PGG formulations have been shown to form a gel under certain conditions. The conditions, such as concentration, during formation of the gel influence the resulting gel properties. In some embodiments, the PGG gel can be formulated to dissolve around 37° C., the body temperature of a patient. Additionally or alternatively, PGG can be formulated as a gel that remains its gel form at around 37° C. or higher temperatures. The gel forms PGG can be used as drug delivery vehicle, for example, a slow release delivery vehicle, with properties adjusted as desired. The gel form PGG can also be used in combination with other delivery systems such as hydrogel and/or poly(lactic-co-glycolic acid) nanoparticles to provide release profiles for a relatively short or extended period.

### Devices for Delivery of Stabilization Compositions

Devices can be adapted for the localized delivery of stabilization compositions to vulnerable plaque. In particular, it can be desirable to deliver stabilization compositions to an isolated section of a blood vessel in the vicinity of identified vulnerable plaque. Through the isolation of a section of a blood vessel, greater control can be maintained over the treatment process, and systemic release of stabilization compositions can be reduced. Further, sequential steps can be performed more directly on the same section of isolated blood vessel. Thus, sequential collagen stabilization and elastin stabilization steps can be performed, rinses can be performed, and the like. Devices have been developed to isolate section of blood vessels with the capability of exchanging fluids with the isolated portions of the vessel. In particular, percutaneous procedures can provide access to the interior of blood vessels. To provide more effective treatment of a selected region of the interior of a blood vessel, regions of the blood vessel can be isolated for treatment using appropriate device. In particular, the delivery devices described in the '833 application and the '311 application, above, provide for the introduction into a blood vessel using less invasive procedures. Furthermore, in some embodiments a profusion balloon can be used. As used herein, a profusion balloon refers to an expandable balloon that leaks fluid from the balloon, in which the leaked fluid can be a stabilization fluid or a treatment fluid.

With respect to devices described in the '833 application, a sealing element of the device can be positioned near the location for treatment of vulnerable plaque within a vessel such that when deployed the sealing element isolates the selected section of the vessel. The delivery device can comprise a single sealing component shaped to form seals at the respective ends of the section of isolated vessel or a plurality of sealing components to for the appropriate seals with the vessel wall. In general, as illustrated in FIG. 2, the extended configuration of an embodiment of an isolation/delivery device is shown. The representative isolation/delivery device 150 comprises a shaft 152 and an extendable element 154. The shaft comprises two lumens 156, 158 that are in fluid communication with the extendable element 154. The device has an optional flexible guide wire like structure 160 to provide for directing into a vessel. At or near the proximal end of the shaft, the lumens of shaft 152 are in fluid communication with ports that are connectable, respectively to liquid delivery/removal devices.

Extendable element 154 comprises a distal balloon 170, a proximal balloon 172, a fluid exchange portion 174, and a by-pass channel 176. Lumen 156 is in fluid communication with balloons 170, 172 so that the inflation and deflation of the balloons can be controlled with fluid flowed through lumen 156. The fluid exchange portion 174 has a plurality of openings 178 that are in fluid communication with lumen 158. In some embodiments, openings 178 of fluid exchange portion 174 can be replaced or supplemented with alternative liquid permeable structures. When deployed in a vessel, balloons 170, 172 form an isolated volume inside the vessel, and fluid flow into the isolated volume can be controlled through fluid exchange portion 174. An alternative device for isolating a section of a blood vessel is described in published U.S. patent application 2001/0029349A to Leschinsky, entitled “Method and Apparatus for Treating Aneurysm,” incorporated herein by reference.

A device for sealing two ends of a selected segment of a vessel with an adjustable length is described in the '311 application. The device generally comprises an introducer sheath with a first sealing element, e.g., a balloon, and a sealing catheter with a second sealing element. The relative positioning of the first sealing element and the second sealing element provides for the selection of the desired volume of the vessel to be isolate. Improved designs of the extendable element of the sealing catheter such as a sealing balloon for placement in the aorta provide for good sealing properties with reduced risk of damaging potentially vulnerable vessel tissue. Further details of the adjustable devices can be found in the '311 application.

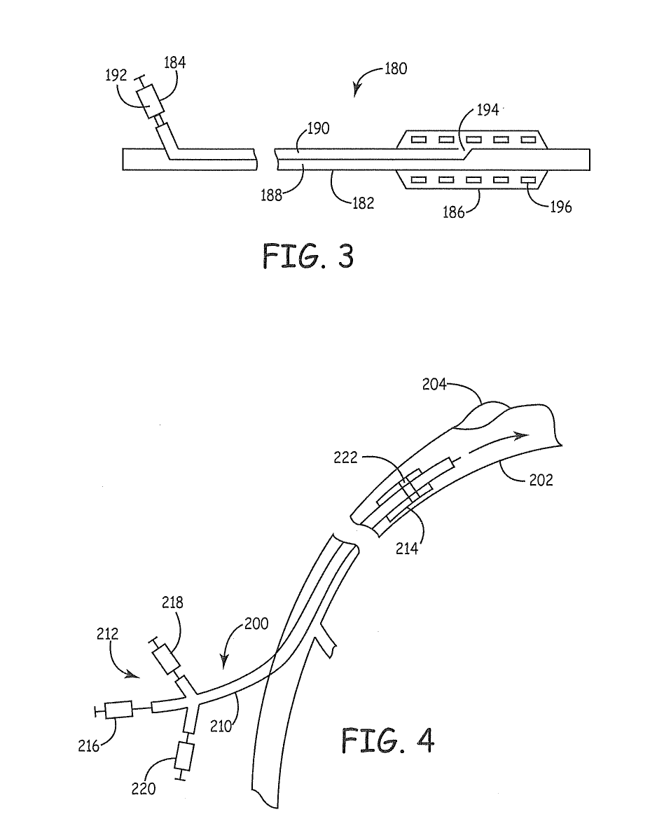

An embodiment of a profusion balloon is shown schematically in FIG. 3. Referring to FIG. 3, profusion balloon 180 comprises shaft 182, fluid delivery element 184 operably connected to shaft 182, and leaky balloon 186 supported on shaft 182. Shaft 182 comprises a guide lumen 188 and balloon lumen 190. Fluid delivery element 184 comprises a fluid reservoir 192 and appropriate fittings to provide a connection with balloon lumen 190. Fluid delivery element can be, for example, a syringe with appropriate Leur fittings or the like to provide a connection of the syringe with the catheter. Balloon lumen 190 comprises a distal port 194 that connects balloon lumen 190 with the interior of balloon 186. Leaky balloon 186 is attached to shaft 182 to fault an interior of the balloon and comprises openings, e.g., punctures or windows, 196 that provide for gradual leaking of fluid out of the balloon. If openings 196 are windows, the window can comprise a material over the window that is slightly porous to provide for gradual leaking. Thus, fluid supplied from reservoir 192 flows through balloon lumen 190 to balloon 186 which inflates with the fluid due to constraints on the leaking of the fluid, and the fluid gradually leaks through the surface of the balloon and the adjacent surroundings. Pressure can be maintained in the balloon through the replacement of fluid that leaks from the balloon after deployment.

### Procedures for Stabilization of Vulnerable Plaque

In general, the stabilization of vulnerable plaque comprises identifying locations in a vessel of vulnerable plaque, positioning a device at a position in the vessel associated with vulnerable plaque and delivering a stabilization composition to the vulnerable plaque. In some embodiments, the device can isolate a section of the vessel including the vulnerable plaque. Once the selected portion of the vessel is appropriately isolated, one or more fluids can be delivered to the isolated portion of the vessel and, if desired, fluids can be removed from the vessel. In alternative or additional embodiments, a profusion balloon can be used to deliver a stabilization composition adjacent the vessel wall.

Several techniques can be adapted to identification of vulnerable plaque. With vulnerable plaque a complication related to detection is that the vulnerable plaque may not be associated with extreme levels of stenosis of the vessels. Some non-invasive techniques can be used in the detection of vulnerable plaque. Non-invasive magnetic resonance imaging (MRI) and multi-slice spiral computed tomography (MSCT) have an excellent ability to identify lipid rich tissue to characterize potentially vulnerable plaques in non-moving structures, such as carotid arteries. MRI and MSCT cannot presently assess small plaque structures due to their resolution. Optical coherence tomography is an alternative technique generally based on near-infrared light. Optical coherence spectroscopy can provide images with ultrahigh resolution using the back reflection of near-infrared light from optical interfaces in tissue. Optical coherence tomography for vulnerable plaque detection is described further in published U.S. patent application 2007/0260138 to Feldman et al., entitled “Optical Coherence Tomographic Detection of Cells and Killing of the Same,” incorporated herein by reference. Raman spectroscopy can provide detailed information about the molecular composition of the plaque, but long acquisition times, low penetration into the tissue and light absorption by blood complicate the performance of Raman spectroscopy in blood vessels. Near infrared spectroscopy is being tested for the identification of lipid loaded plaques.

The labeling of inflammatory factors associated with vulnerable plaque can also be used for detection of vulnerable plaque. The use of biomarkers for identification of vulnerable plaque is described in published U.S. patent application 2007/0207507 to Kim et al., entitled “Biomarkers of Vulnerable Plaques and Methods of Use,” incorporated herein by reference. In the proposed biomarker approach, the biomarkers become associated with the vulnerable plaque, and then the biomarkers are detected with non-invasive imaging techniques. The biomarkers can be labeled with super-paramagnetic iron oxide or gadolinium to provide for detection using MRI imaging or a near-infrared florescence label for detection using fluorescence mediated tomography.

Angiography involves the injection of contrast dye into the blood vessels and imaging using x-ray techniques. Angiography has a low discriminatory power to identify the vulnerable plaque, but this technique provides information about the entire coronary tree and serves as a guide for additional imaging techniques and for invasive therapy. Angiography offers a direct visualization of the plaque surface and intra-luminal structures, such as thrombi and tears. Angiography can be effective in particular for proximal portions of the vessel.

Thermography assesses the temperature heterogeneity as an indicator of the metabolic state of the plaque. A coincidence of temperature rise and localization of vulnerable plaque has been proposed based on the presence of dense macrophage infiltration. The temperature rise is generally inversely related to cap thickness. The use of thermography for vulnerable plaque detection is described further in U.S. Pat. No. 7,288,244 to Van Langenhove et al., entitled “Determining Vulnerable Plaque in Blood Vessels,” incorporated herein by reference.

Intravascular ultrasound can be used in vulnerable plaque evaluation. Intravascular ultrasound can provide some insight into the composition of plaques. A device for intravascular ultrasound imaging is described in U.S. Pat. No. 7,226,417 to Eberle et al., entitled “High Resolution Intravascular Ultrasound Transducer Assembly Having a Flexible Substrate,” incorporated herein by reference. The specific use of intravascular ultrasound for vulnerable plaque characterization is described further in published U.S. patent application 2009/0299186 to Waters et al., entitled “System and Method for Characterizing Tissue Based upon Homomorphic Deconvolution of Backscattering Ultrasound,” incorporated herein by reference.

To provide the stabilization of the vulnerable plaque, a section of blood vessel can be selected for treatment. A suitable intravascular device can then be placed within the blood vessel using a percutaneous procedure, for example, using known hemostatic procedures, hemostatic valves and the like. In some embodiments, the device can be deployed to seal the selected section of the blood vessel. A portion of the blood can then be removed from the isolated section of vessel if desired to facilitate delivery of a stabilization fluid. A liquid can be delivered simultaneously or intermittently with the removal of blood to maintain a reasonable internal pressure within the vessel.

Based on the identified location of the vulnerable plaque, an appropriate delivery device, such as the devices described herein, can be used for delivery of one or more stabilization compositions that can comprise VEGF, a collagen stabilization agent and/or an elastin stabilization agent that are targeted to a specific site. The delivery device can be placed into the blood vessel using a less invasive procedure to provide delivery of the stabilization agent locally from the interior of the vessel. Percutaneous entry into blood vessels and corresponding delivery technologies for catheter use for vascular procedures suitable for introducing the devices described herein are generally known to those of skill in the art, and can be adapted for use with the devices for the delivery of therapeutic compositions described herein. Delivery of the stabilization agent using the delivery devices can be supplemented with the use of a stent, which may or may not further comprise a drug coating. U.S. Pat. No. 6,979,347 to Wu, et al., incorporated herein by reference, describes an apparatus and associated method for delivering a therapeutic substance through a coating on a stent, such as the genipin. Furthermore, an angioplasty balloon can be used to mechanically reduce stenosis at the location of the vulnerable plaque before and/or after delivery of a stabilization composition.

A percutaneous procedure for the delivery a stabilization composition at the location of vulnerable plaque in a blood vessel, generally an artery, is shown in FIG. 4. Delivery device 200 is brought into vessel 202 to provide access to vulnerable plaque 204. Delivery device 200 comprises tubular shaft 210, proximal end 212 and sealing element 214. As shown in the embodiment of FIG. 4, proximal end 212 comprises an expansive fluid delivery element 216, a suction element 218 and stabilization fluid delivery element 220. Expansive fluid delivery element 216, which can comprise a syringe or the like, comprises a fluid, such as sterile saline, to expand and subsequently deflate sealing element 214. Suction element 218 can comprise a syringe or other negative pressure device that is unpowered or powered and is in fluid communication with a lumen extending through tubular shaft 210 to sealing element 214. Stabilization fluid delivery element 220, which can comprise a syringe, pump or the like, comprises a reservoir of a stabilization composition that can be delivered through tubular shaft 202 to sealing element 214. Tubular shaft 210 comprises an appropriate number of distinct lumens to provide for fluid communication between proximal end 212 and sealing element 214 such that the expansion and deflation sealing element 214 can be separately controlled relative to the delivery and removal of fluid from a isolated segment of the vessel.

Sealing element 214 comprises one or more balloons or other expansive elements such that the sealing element can be expanded to isolate a portion of the vessel. Sealing element 214 comprises a fluid exchange element 222. Fluid exchange element 222 provides for the exchange of fluid to and from an isolated volume between the wall of the sealing element and the vessel wall within the isolated portion of the vessel.

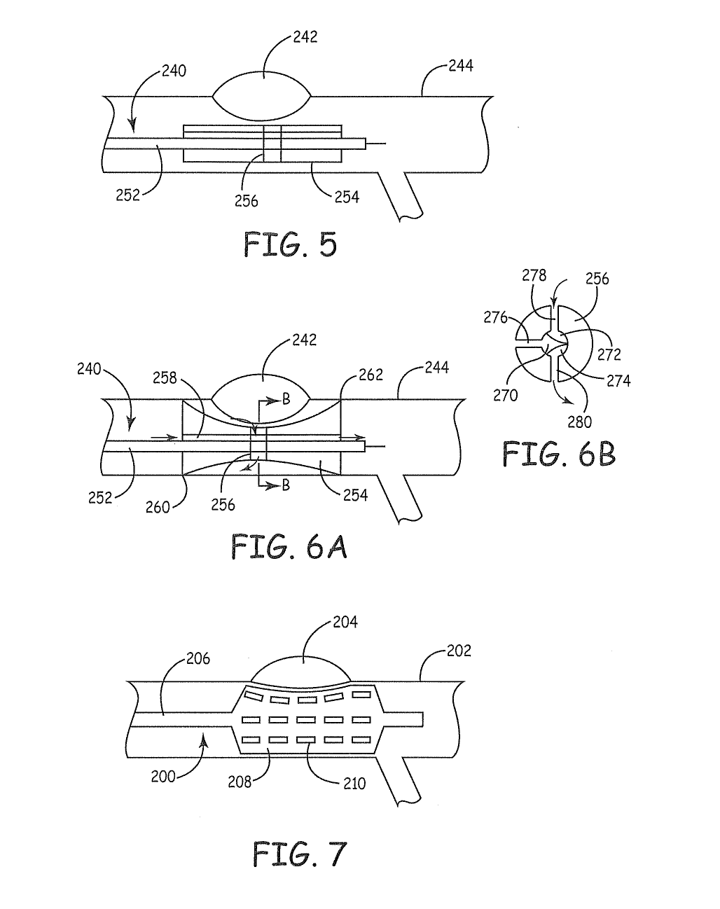

Referring to FIG. 5, a representative embodiment of a delivery device 240 is shown prior to deployment at the position of vulnerable plaque 242 within vessel 244. Delivery device 240 is shown in a deployed configuration in FIG. 6A. Delivery device 240 comprises shaft 252, balloon element 254, fluid exchange element 256 and flow by-pass channel 258. In the expanded configuration, balloon element forms a sealed engagement with vessel 244 at edges 260, 262. Flow by-pass channel 258 provided for flow of blood past sealed edges 260, 262 as shown with the flow arrows. Referring to FIG. 6B, fluid exchange element 256 interfaces with a balloon lumen 270, an aspiration lumen 272 and fluid delivery lumen 274 with respective channels, 276, 278, 280. Lumen 270, 272, 274 extend through shaft 252 to a proximal portion exterior to the patient so that fluid movement can be appropriately controlled by a health care professional during use of the device. Balloon lumen 270 provides for flow of a suitable fluid through channel 276 into and out from balloon 254 to provide for expansion and collapse of the balloon. Aspiration lumen 272 provides for the removal of fluid from the isolated portion of the vessel between sealed edges 260, 262 with the fluid flowing through channel 278. Fluid delivery lumen 274 and channel 280 provides for the delivery of a stabilization composition and/or therapeutic agents into the isolated portion of vessel 244.

In additional of alternative embodiments, a profusion balloon can be used to deliver a stabilization composition, a therapeutic compositions, or combinations thereof. A deployed leaky balloon within a blood vessel is shown schematically in FIG. 7. Perfusion balloon catheter 200 is located in vessel 202 at a location of vulnerable plaque 204. Profusion balloon catheter 200 comprises a shaft 206 with a balloon lumen and a leaky balloon 208. Leaky balloon 208 comprises windows 210 through the body of the balloon. When balloon 208 is inflated with fluid delivered through the balloon lumen, the balloon can apply mechanical force against the vulnerable plaque 204 to at least partially decrease constriction of vessel 202 at the vulnerable plaque. A stabilization composition and/or a therapeutic composition can then also leak from balloon 208 at windows 210 for delivery at the wall of vessel 202.

The procedures generally may vary in some respects based on the nature of the sealing element. For example, devices with two or more flow lumen can provide for aspiration through one lumen and delivery of fluids through another lumen, while a single lumen device correspondingly involves sequential approaches for the delivery and removal of fluids. In some embodiments, additional steps of delivering and removing liquids from the isolated region can be performed if desired, such as for the sequential contact with multiple stabilization fluids and/or fluids comprising therapeutic agents. In some embodiments, the step of delivering the stabilization composition is repeated with a different composition, wherein the delivery of one stabilization composition comprises the delivery of a collagen stabilization composition and the delivery of another stabilization composition comprises the delivery of an elastin stabilization composition. The order of the delivery agents can be selected as desired.

A first therapeutic composition can be delivered through a flow passage of the sealing device to the isolated portion of the vessel to provide stabilization of the vulnerable plaque. The therapeutic composition can be left in the vessel for a suitable period of time to affect the vessel wall in desired ways. In some embodiments, a therapeutic composition can be delivered into the vessel for at least about 1 minute, in further embodiments, for at least about 2 minutes, in additional embodiments for a period from about 2.5 minutes to about 2 hours, and from about 3 minutes to about 1 hour. A person of ordinary skill in the art will recognize that additional ranges of times within the explicit ranges above are contemplated and are within the present disclosure.

After treatment for an appropriate period of time, the therapeutic fluid can be removed through the same or a different flow passage associated with the sealing device. The therapeutic fluid can be replaced with a second therapeutic fluid, a rinse fluid, and/or blood, although the seal can also be deflated to allow for blood from the vessel into the previously isolated section of the vessel. Suitable rinse fluids can comprise buffered saline or the like. In some embodiments, a suitable rinse fluid comprises agents that reduce residual toxicity, especially for aldehyde crosslinked tissue. Also, dilute ethanol can be used as a rinse agent, and dilute ethanol has been used to rinse glutaraldehyde crosslinked bioprosthetic tissue for human use. Suitable rinse agents are described further above. The blood can be replacement of blood from the patient or other compatible blood. A second therapeutic fluid can be any appropriate therapeutic fluid described herein, and further treatment fluids and/or rinsing fluids can be similarly administered, as desired, in any reasonable combination.

In some embodiments, a first treatment fluid comprises a protein crosslinking agent. The protein crosslinking agent can strengthen the vulnerable plaque to reduce the risk of rupturing plaque. A second treatment fluid can comprise VEGF, optionally with a suitable concentration of protein crosslinking agent. It has been demonstrated that VEGF can be associated with a collagen containing material using a relatively low level of crosslinking agent while maintaining the function of the VEGF. See published PCT application, WO 99/37337 to Carlyle et al., entitled “Prostheses With Associated Growth Factor,” incorporated herein by reference. After contact with the VEGF solution for an appropriate period of time, the residual VEGF solution can be removed, and optionally replaced with another solution, such as a rinsing solution. After desired manipulations have been completed within the isolated portion of the vessel, the sealing elements can be collapsed to a recovery configuration, and the device can be removed from the patient.

The chemical treatment approaches described herein can provide a decreased risk of plaque fracture and an improvement in the health of the tissue. However, these techniques are not particularly designed to decrease occlusion of a vessel which may have become partially occluded due to plaque build up. Thus, the treatment of an isolated region described herein can be performed following an angioplasty procedure, the delivery of a stent, an athorectomy procedure or the like. Suitable procedures include, for example conventional procedures designed to open up a blood vessel or yet to be developed procedures. Filtration or other embolic protection can accompany the procedure. After the procedure to relieve or reduce the vascular occlusion, the therapeutic procedures within the isolated section of the vessel described above can be performed to stabilize the vessel.

The embodiments above are intended to be illustrative and not limiting. Additional embodiments are within the inventive concepts. In addition, although the present invention has been described with reference to particular embodiments, those skilled in the art will recognize that changes can be made in form and detail without departing from the spirit and scope of the invention. Any incorporation by reference of documents above is limited such that no subject matter is incorporated that is contrary to the explicit disclosure herein. All patents, patent applications, and publications referenced herein are hereby incorporated by reference herein to the extent that the incorporated material is not contrary to any of the explicit disclosure herein.

What is claimed is:

1. A method for the treatment of vulnerable plaque comprising:
delivering a first stabilization composition to stabilize vulnerable plaque within an isolated section of a blood vessel at a location including identified vulnerable plaque,
wherein the composition comprises an elastin stabilization agent, a collagen stabilization agent, VEGF, or a combination thereof.

2. The method of claim 1 further comprising isolating the section of the blood vessel identified as having a clinically significant degree of vulnerable plaque using a device that can be delivered into the vessel in a less invasive format.

3. The method of claim 2 wherein the device seals the vessel at two locations while providing exchange of fluids with the isolated section of the vessel using a lumen that extends through a catheter shaft to the exterior of the patient's vessel.

4. The method of claim 3 wherein the device provides for the profusion of blood past the isolated section of vessel.

5. The method claim 1 wherein identification of the location is performed with intravascular ultrasound.

6. The method of claim 1 wherein the first stabilization composition comprises an elastin stabilization agent and wherein the elastin stabilization agent comprises PGG.

7. The method of claim 1 wherein the first stabilization composition comprises a collagen stabilization agent selected from the group consisting of a difunctional aldehyde, diamines, genipin, acyl azide, epoxyamines, or combinations thereof.

8. The method of claim 1 further comprising delivering a second stabilization composition to the location.

9. The method of claim 8 wherein the first stabilization composition comprises glutaraldehyde and the second stabilization composition comprises VEGF.

10. The method of claim 1 further comprising removing at least a portion of the first stabilization composition after passage of a selected period of time.

11. The method of claim 10 further comprising delivering a rinse composition to the isolated portion of the vessel after the removal of the at least a portion of the first stabilization composition.

12. A composition comprising a biocompatible carrier, a collagen crosslinking agent and a VEGF growth factor, wherein composition has a concentration of the collagen crosslinking agent at least about 0.5 w/v %.

13. The composition of claim 12 wherein the collagen crosslinking agent comprises glutaraldeyhde.

14. The composition of claim 12 having a concentration of VEGF from about from about 1 ng/ml to about 10 μg/ml.

15. A method for stabilizing vulnerable plaque in an artery, the method comprising delivering the composition of claim 12 to the vulnerable plaque.

16. The method of claim 15 wherein the composition is formed during the delivery process.

17. A medical device for delivery of a composition at a location in a blood vessel, the device comprising: a shaft comprising a balloon lumen, a permeable balloon supported by the shaft with an interior in fluid communication with the balloon lumen, and a reservoir holding a stabilization agent near the distal end of the shaft and being configured for controlled delivery of the stabilization agent to the balloon through the balloon lumen, wherein the stabilization agent comprises an elastin stabilization agent, a collagen stabilization agent, VEGF, or a combination thereof.

18. The medical device of claim 17 wherein the stabilization agent comprises VEGF.

19. A method for the stabilization of vulnerable plaque, the method comprising using a medical device of claim 17 to deliver the stabilization agent in the vicinity of the vulnerable plaque wherein the permeable balloon is placed in the vessel at or near the vulnerable plaque.

20. A medical device comprising a vascular isolation apparatus and a reservoir of VEGF, wherein the vascular isolation device comprises a sealing element operably connected with a catheter having a lumen with fluid passage between the reservoir and a fluid delivery port, the sealing member being configured for isolating a portion of a blood vessel when the catheter is delivered in a less invasive endovascular procedure, and wherein the fluid delivery port is positioned for delivery of fluid to the isolated portion of the blood vessel.

21. The medical device of claim 20 further comprising a passage for the profusion of blood past the isolated portion of the vessel.

22. The medical device of claim 20 wherein the sealing element comprises two distinct balloons that are configured to seal the respective ends of the isolated portion of the vessel.

23. The medical device of claim 20 wherein the device comprises a single extendable element that is shaped to isolated the selected location.
